(19) United States
(12) Patent Application Publication
(10) Pub. No.: US 2014/0296972 A1
(43) Pub. Date: Oct. 2, 2014

(54) Deployment Compensator for Transcatheter Valve Delivery

(71) Applicants: Zachary Tegels, Minneapolis, MN (US); Robert VIDLUND, Forest Lake, MN (US)

(72) Inventors: Zachary Tegels, Minneapolis, MN (US); Robert VIDLUND, Forest Lake, MN (US)

(73) Assignee: Tendyne Holdings, Roseville, MN (US)

(21) Appl. No.: 14/255,691

(22) Filed: Apr. 17, 2014

Related U.S. Application Data
(60) Provisional application No. 61/807,694, filed on Apr. 2, 2013.

(51) Int. Cl. A61F 2/24

(52) U.S. Cl. CPC A61F 2/2427
USPC 623/2.11

(57) ABSTRACT

This invention relates to a deployment compensator for reducing the expelling force of a compressed transcatheter valve while it is being deployed in a patient in need thereof, and methods of use thereof.

### CROSS REFERENCE TO RELATED APPLICATIONS

Not applicable

### STATEMENT REGARDING FEDERALLY SPONSORED RESEARCH OR DEVELOPMENT

No federal government funds were used in researching or developing this invention.

### NAMES OF PARTIES TO A JOINT RESEARCH AGREEMENT

Not applicable.

### SEQUENCE LISTING INCLUDED AND INCORPORATED BY REFERENCE HEREIN

Not applicable.

### BACKGROUND

1. Field of the Invention

This invention relates to relates to a deployment compensator for reducing the expelling force of a compressed transcatheter valve while it is being deployed in a patient in need thereof, and methods of use thereof.

2. Background of the Invention

Valvular heart disease and specifically aortic and mitral valve disease is a significant health issue in the US Annually approximately 90,000 valve replacements are conducted in the US. Traditional valve replacement surgery, the orthotopic replacement of a heart valve, is an “open heart” surgical procedure. Briefly, the procedure necessitates surgical opening of the thorax, the initiation of extra-corporeal circulation with a heart-lung machine, stopping and opening the heart, excision and replacement of the diseased valve, and re-starting of the heart. While valve replacement surgery typically carries a 1-4% mortality risk in otherwise healthy persons, a significantly higher morbidity is associated to the procedure largely due to the necessity for extra-corporeal circulation. Further, open heart surgery is often poorly tolerated in elderly patients.

Thus if the extra-corporeal component of the procedure could be eliminated, morbidities and cost of valve replacement therapies would be significantly reduced.

While replacement of the aortic valve in a transcatheter manner is the subject of intense investigation, lesser attention has been focused on the mitral valve. This is in part reflective of the greater level of complexity associated to the native mitral valve apparatus and thus a greater level of difficulty with regards to inserting and anchoring the replacement prosthesis.

Several designs for catheter-deployed (transcatheter) aortic valve replacement are under various stages of development. The Edwards SAPIEN transcatheter heart valve is currently undergoing clinical trial in patients with calcific aortic valve disease who are considered high-risk for conventional open-heart valve surgery. This valve is deployable via a retrograde transarterial (transfemoral) approach or an antegrade transapical (transventricular) approach. A key aspect of the Edwards SAPIEN and other transcatheter aortic valve replacement designs is their dependence on lateral fixation (e.g. tines) that engages the valve tissues as the primary anchoring mechanism. Such a design basically relies on circumferential friction around the valve housing or stent to prevent dislodgement during the cardiac cycle. This anchoring mechanism is facilitated by, and may somewhat depend on, a calcified aortic valve annulus. This design also requires that the valve housing or stent have a certain degree of rigidity.

At least one transcatheter mitral valve design is currently in development. The Endovalve uses a folding tripod-like design that delivers a tri-leaflet bioprosthetic valve. It is designed to be deployed from a minimally invasive transatrial approach, and could eventually be adapted to a transvenous atrial septotomy delivery. This design uses “proprietary gripping features” designed to engage the valve annulus and leaflets tissues. Thus the anchoring mechanism of this device is essentially equivalent to that used by transcatheter aortic valve replacement designs.

Various problems continue to exist in this field, including problems with insufficient articulation and sealing of the valve within the native annulus, pulmonary edema due to poor atrial drainage, perivalvular leaking around the install prosthetic valve, lack of a good fit for the prosthetic valve within the native mitral annulus, atrial tissue erosion, excess wear on the nitinol structures, interference with the aorta at the posterior side of the mitral annulus, and lack of customization, to name a few. Accordingly, there is still a need for an improved valve having a commissural sealing structure for a prosthetic mitral valve.

### BRIEF SUMMARY OF THE INVENTION

The present invention relates to a deployment compensator for reducing the expelling force of a compressed transcatheter valve while it is being deployed in a patient.

In a preferred embodiment, there is provided a deployment compensator for deploying a transcatheter prosthetic cardiovascular valve in a patient, which comprises an extension spring connecting an end block to a spring head, the end block sized to remain outside of a valve deployment catheter sheath and having a sheath guide block connected thereto for inserting into an end portion of a valve deployment catheter sheath, said end block and sheath guide block having a push rod aperature for mounting a push rod, the spring head having a spring barrel connected thereto, the spring barrel disposed within the extension spring, and said spring head and spring barrel having a push rod aperture for mounting a push rod.

In another preferred embodiment, there is provided a deployment compensator for reducing the expelling force of a compressed transcatheter valve while it is being deployed in a patient in combination a push rod having a collet a a distal end and a removable epicardial attachment pad attached to a proximal end of the push rod.

In another preferred embodiment, there is provided a deployment compensator wherein the device has one or more radio-opaque markers thereon to facilitate positioning.

In another preferred embodiment, there is provided a deployment compensator where the device fits within a surgical catheter sheath having a diameter of between about 10 Fr (3.3 mm) to about 42 Fr (14 mm).

In another preferred embodiment, there is provided a method of reducing the expelling force of a compressed transcatheter prosthetic cardiovascular valve out of a delivery catheter during deployment in a patient, which comprises the step of connecting a valve tether to a deployment compensator as in claim 1 while the valve is being expelled from the delivery catheter being used to surgically deploy the valve into the patient in need thereof

### BRIEF DESCRIPTION OF THE DRAWINGS

FIG. 1 is a side view of a deployment compensator according to the present inventive subject matter.

FIG. 2 are end views showing each end, proximal and distal, of the deployment compensator.

FIG. 3 is a side view showing a standard push rod.

FIG. 4 is a side view showing the deployment compensator attached to a valve/valve tether during the expelling process.

FIG. 5 is a side view of the deployment compensator attached to the valve/valve tether and where the deployment compensator is shown being stretched into an elongated (extended) position and used to reduce the force of the valve as it is expelled from the delivery catheter, thus limiting the distance that the valve travels from the end of the delivery sheath/catheter.

### DETAILED DESCRIPTION OF THE INVENTION

### Functions of the Positioning Tool

When a transcatheter valve is delivered, the compressed valve is expelled from the delivery catheter and the valve expands to its functional structure. In the case of a prosthetic mitral valve that uses an atrial cuff in combination with a ventricular tether to seat itself within the mitral annulus, when the valve is deployed into the left atrium, the valve shoots with great force from the end of the delivery catheter due to the strong compressive force that had been keeping the valve in the delivery catheter. This force is so large that it can cause significant damage to tissue, e.g. left atrium. The deployment compensator is used to pull the valve back towards the delivery catheter using an extension spring and counter-act the expelling force, this avoiding tissue damage.

### Pledget/Attachment Pad

In a preferred embodiment, an epicardial pledget or attachment pad may be integrated directly into the deployment compensator, for instance on the push rod so that once proper deployment is achieved, the pad may be slid into place and surgically secured.

In one embodiment, to control the potential tearing of tissue at the apical entry point of the delivery system, a circular, semi-circular, or multi-part pledget is employed. The pledget may be constructed from a semi-rigid material such as PFTE felt. Prior to puncturing of the apex by the delivery system, the felt is firmly attached to the heart such that the apex is centrally located. Secondarily, the delivery system is introduced through the central area, or orifice as it may be, of the pledget. Positioned and attached in this manner, the pledget acts to control any potential tearing at the apex.

### DESCRIPTION OF FIGURES

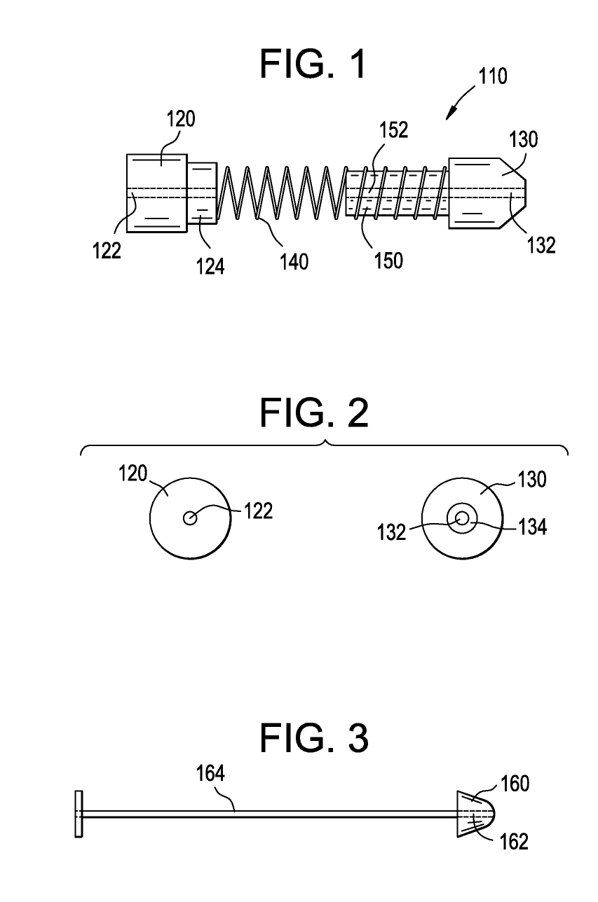

Referring now to the FIGURES, FIG. 1 is a side view of a deployment compensator according to the present inventive subject matter. FIG. 1 shows deployment compensator 110 having extension spring 140 connecting end block 120 and spring head 130. FIG. 1 shows end block 120 having end block push rod aperture 122 and sheath guide 124. Sheath guide 124 fits within the deployment catheter, but end block 120 does not, this providing a stabilizing catheter plug at a proximal end of the delivery catheter. Spring head 130 has spring head aperture 132 and is also connect to barrel 150, which also has barrel push-rod aperture 152, for receiving the push rod (not shown). Spring head aperture 132 operates, in one embodiment, as a tensioning device to allow the valve tether (not shown) to advance through the aperture slowly, but to engage and reduce the travel speed of the tether through the aperture if a large longitudinal force is applied. This allows transference of the force to the extension spring and allows the spring to provide a counter-acting force in the opposite direction.

FIG. 2 are end views showing each end, proximal and distal, of the deployment compensator. FIG. 2 shows end block 120 and end block push-rod aperture 122, and spring head 130, with cooperative surface 132, and spring head push-rod aperture 134.

FIG. 3 is a side view showing a standard push rod. FIG. 3 shows rod 164, collet 160 and rod tether aperture 162.

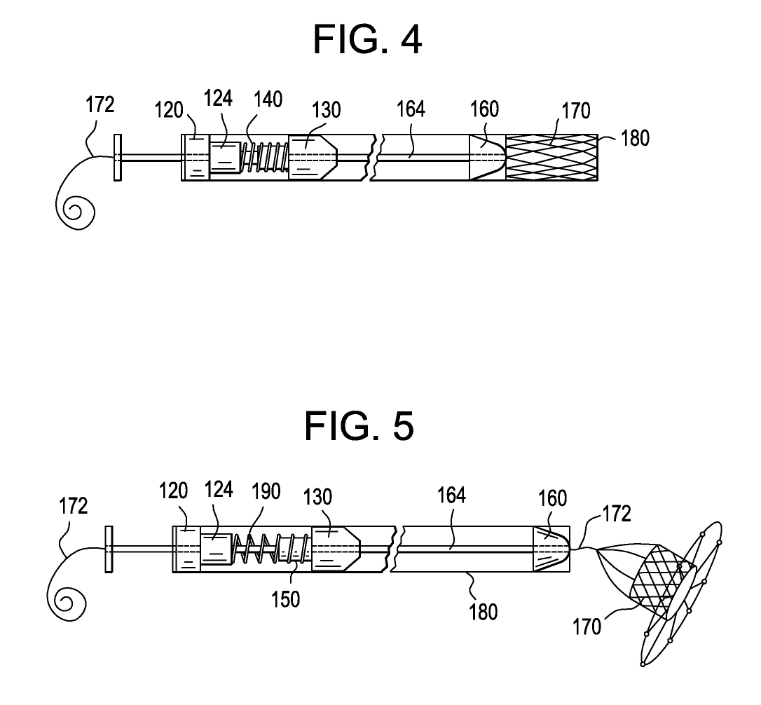

FIGS. 4 and 5 are side views showing the deployment compensator attached to a valve/valve tether 172 during the expelling process. FIGS. 4 and 5 shows end block 120 and sheath guide 124 having tether 172 extending through them. Spring 140 is shown compressed in FIG. 4 and then travels to an extended state in FIG. 5, shown by the spring head moving 130 and barrel 150 moving from left to right, as the push rod 164 and collet 160 move from left to right, expelling the valve 170 from the end of the delivery catheter 180.

FIG. 5 is a side view of the deployment compensator attached to the valve/valve tether 172 and where the deployment compensator is shown being stretched into an elongated (extended) position and used to reduce the force of the valve as it is expelled from the delivery catheter 180, thus limiting the distance that the valve 170 travels from the end of the delivery sheath/catheter 180.

The references recited herein are incorporated herein in their entirety, particularly as they relate to teaching the level of ordinary skill in this art and for any disclosure necessary for the commoner understanding of the subject matter of the claimed invention. It will be clear to a person of ordinary skill in the art that the above embodiments may be altered or that insubstantial changes may be made without departing from the scope of the invention. Accordingly, the scope of the invention is determined by the scope of the following claims and their equitable Equivalents.

What is claimed is:

1. A deployment compensator for deploying a transcatheter prosthetic cardiovascular valve in a patient, which comprises an extension spring connecting an end block to a spring head, the end block sized to remain outside of a valve deployment catheter sheath and having a sheath guide block connected thereto for inserting into an end portion of a valve deployment catheter sheath, said end block and sheath guide block having a push rod aperature for mounting a push rod, the spring head having a spring barrel connected thereto, the spring barrel disposed within the extension spring, and said spring head and spring barrel having a push rod aperture for mounting a push rod.

2. The deployment compensator of claim 1, further comprising in combination a push rod having a collet a a distal end and a removable epicardial attachment pad attached to a proximal end of the push rod.

3. The deployment compensator of claim 1, wherein the device has one or more radio-opaque markers thereon to facilitate positioning.

4. The deployment compensator of claim 1, where the device fits within a surgical catheter sheath having a diameter of between about 10 Fr (3.3 mm) to about 42 Fr (14 mm).

5. A method of reducing the expelling force of a compressed transcatheter prosthetic cardio-vascular valve out of a delivery catheter during deployment in a patient, which comprises the step of connecting a valve tether to a deployment compensator as in claim 1 while the valve is being expelled from the delivery catheter being used to surgically deploy the valve into the patient in need thereof.
